(19) United States
(12) Patent Application Publication
(10) Pub. No.: US 2015/0231112 A1
(43) Pub. Date: Aug. 20, 2015

(54) NASAL AND SINUS WASH COMPOSITIONS AND METHODS

(71) Applicant: Brian Stanislaws Paul, Los Angeles, CA (US)

(72) Inventor: Brian Stanislaws Paul, Los Angeles, CA (US)

(21) Appl. No.: 14/181,398

(22) Filed: Feb. 14, 2014

(51) Int. Cl. A61K 31/375

(52) U.S. Cl. CPC A61K 31/375

(57) ABSTRACT

Nasal and sinus cavity wash compositions containing magnesium ascorbate and, optionally, a bicarbonate salt, are provided to cleanse the nasal and sinus cavities of a subject and to reduce nasal and sinus irritation, including inflammation.

### FIELD OF THE INVENTION

The present invention relates to nasal and sinus wash compositions, and methods of their use to cleanse nasal and sinus cavities and to reduce nasal and sinus irritation.

### BACKGROUND OF THE INVENTION

The mucous membranes inside the nose are sensitive to substances that enter from the environment and may become inflamed from a variety of substances, resulting in swelling, congestion, pain, sneezing and other symptoms. It is estimated that 20-30 percent of the population, in the United States alone, suffers from allergies of the nasal and sinus mucosa.

Allergic rhinitis, also called “hay fever” or “seasonal allergy,” involving both nasal sinus and mucosa, includes a group of symptoms affecting the nose and results from inflammation of the mucous membranes. These symptoms occur when an individual inhales an “allergen,” such as dust, mold, dust, animal dander, or pollen. In response, chemicals may be released by the body, which may further irritate nasal passages. Symptoms can also occur when a food allergen is consumed. Allergy sufferers experience symptoms ranging from mild irritation, for example “itchiness,” to severe symptoms such as a stuffy or runny nose, sneezing, coughing, blocked and inflamed nasal passages, difficulty breathing, blocked eustacian tubes, that can lead to infection and sinusitis, and watery, itchy eyes. Additional worsening symptoms include a decreased sense of smell, a feeling of “clogged” ears, sore throat, dark circles, and/or puffy skin under the eyes, fatigue, general irritability and headache. More serious symptoms can also result.

In allergic rhinitis and asthma, the intracellular calcium levels have been shown to increase, as a result of IgE stimulation, causing histamine release from mast cells. Studies show that nasal irrigation may help ameliorate, lessen the duration, or even prevent episodic, perennial, and chronic sinus conditions including, for example, dryness, rhinitis, and sinusitis. Studies have also demonstrated the benefits of nasal irrigation in speeding recovery from nasal and sinus surgery. (Rakei, D., (2012) Integrative Medicine, Third Edition, Elsevier; Rabag, D., Zgierska, A., (2006) Am. Fam. Physician (2009), 80:1117-1119; Rabago, D., Annals of Family Medicine, (2006) 4:295-301); and Rabago, D. et al., (2002) J. Fam. Pract. 51:1049-1055).

The terms “nasal irrigation,” “nasal lavage,” “sinus wash,” and “nasal wash” generally refer to the introduction of a solution, for example a simple saline solution, into the nasal and sinus cavities, for the purpose of cleansing, medicating, moisturizing irritated sinus and nasal cavities and membranes. This procedure has become more commonly accepted in daily hygiene routines. Irrigation may be performed manually with simple gravity or by manual pressurization of the saline solution through use of a squeeze bottle. Additionally, modified electric irrigators have also been used in sinus washing.

Solutions for use in nasal and sinus washing include isotonic solutions of saline (0.9% sodium chloride) and buffering agents to maintain the solution at a basic pH. Isotonic solutions prevent osmotic effects and consequent damage to cells. Bicarbonate, due in part to its major role in mammalian physiology and favorable buffering characteristics, has been used as a buffering agent in nasal wash solutions. Bicarbonate is a major constituent of blood and helps to maintain a basic pH of about 7.4. A blood pH of less than about 6.8 may lead to cell damage and even death. In the sinuses, the pH may vary between about 5 to about 8, but is generally about 6.4. Potassium bicarbonate has shown anti-fungal properties and reduction of the fungal allergic response. (deShazo R D. et al., 1997, N. Engl. J. Med: 337:254-259).

Studies have shown redaction in allergic nasal symptoms using ascorbic acid (Vitamin C). (Murray M T. A comprehensive review of vitamin C. Am J. Nat Med 1996, 3:8-21); Helms and Miller, Alternative Medicine Review, 10(4) (December 2005).

Bromelain is a proteolytic enzyme complex flavonoid that when provided orally has shown anti-inflammatory properties and thinning of mucus. (Lotz-Winter H, “On the pharmacology of bromelain: an update with special regard to animal studies on close-dependent effects”. Planta Med. 56 (3): 249-53 (June 1990). Quercetin (2-(3,4-dihydroxypbenyl)-3,5,7-trihydroxy-4H-chromen-4-one) is a plant derived flavonoid molecule shown to have anti-inflammatory activity (Park, H J et al/. Int. Immunopharmacol. 9 (3):261-7 (2009).

Magnesium has been demonstrated to reduce calcium increases that result in IgE stimulation and cause histamine release from mast cells, and to function as a bronchodilator, (Hill et al., 1997 Clinical Exp. Allergy 27: 546-511: Rolla et al., 1987 Allergy 42:186-8.

There remains a need for nasal and sinus washes which contain components that cleanse the nasal and sinus cavities, and to address nasal and sinus irritation.

### SUMMARY OF THE INVENTION

Accordingly, the present invention provides a liquid nasal and sinus wash composition containing magnesium ascorbate. The wash composition may include a bicarbonate salt, that may be potassium bicarbonate, and may further include a flavonoid, which can be quercetin or bromelain.

An embodiment of the invention is a dry composition for preparing a nasal and sinus wash that includes approximately 50% by weight up to approximately 98% by weight of magnesium ascorbate, and from approximately 50% by weight down to approximately 2% by weight of a bicarbonate salt. The bicarbonate sale of the composition may be potassium bicarbonate, and may also include up to approximately 5% by weight of a flavonoid, that may be quercetin or bromelain.

An embodiment of the invention is a nasal and sinus wash solution containing magnesium ascorbate and water. The solution can also include potassium bicarbonate. The solution includes approximately 2.9% to approximately 5.68% by weight of magnesium ascorbate, and from approximately 0.025% to approximately 0.7% by weight of potassium bicarbonate. The pH of the solution is from approximately 6.0 to approximately 8.0, or from from approximately 6.8 to approximately 7.1. The solution contains approximately 50 grams of magnesium ascorbate and approximately 2 grams of potassium bicarbonate in 1 liter of sterilized or purified water. The solution may also include approximately 2 grams of quercetin. The solution can be isotonic, hypertonic or hypotonic, relative to human or animal cells.

Another embodiment of the invention is a method of cleansing nasal and sinus cavities and reducing symptoms of nasal and sinus irritation, including inflammation, in a subject, by administering an effective amount of the wash solution of the invention into the nasal and sinus cavity of a subject.

### DETAILED DESCRIPTION OF THE INVENTION

All publications cited herein are hereby incorporated by reference in their entirety.

As used herein, the term “nasal” refers to a nose and nasal passages in an individual, and “sinus” refers to the paranasal sinuses that communicate with the nasal cavity. The terms “cavity” and “passages” are used interchangeably to connote the internal space within the nose or sinuses of an individual. The term “cleanse” means to remove irritants, including debris, from the nasal and sinus cavities of a subject.

The compositions of the present invention contain an effective amount of magnesium ascorbate and, optionally an effective amount of a bicarbonate salt such as potassium or sodium bicarbonate, and an effective amount of a flavonoid such as quercetin or bromelain, to cleanse and to reduce the irritation of nasal and sinus cavities of a subject. The compositions may be prepared in dry (i.e. powder) form, and are preferably mixed with water to form a solution, prior to administration to a human or animal subject. Human cells have a tonicity of from approximately 285-300 milliosmols. The solutions of the invention may be isotonic relative to human or animal cells, hypertonic relative to human or animal cells, being equal to or greater than 300 milliosmols, or may be hypotonic, being equal to or less than approximately 285 milliosmols. The water used to make the wash solutions of the invention may be pre-treated, for example, by distillation, de-ionization, filtration, or a combination thereof.

In an embodiment of the compositions of the invention a dry composition contains 100% magnesium ascorbate.

In another embodiment of the compositions of the invention, the dry composition includes from approximately 50% by weight up to approximately 98% by weight of magnesium ascorbate, and from approximately 50% by weight down to approximately 2% by weight of a bicarbonate salt, such as potassium bicarbonate. The formulation can include up to approximately 5% by weight of a flavonoid, such as quercetin or bromelain.

One embodiment of 3.75 grams of a dry composition of the invention contains approximately 2 grams of magnesium ascorbate, and approximately 0.75 grams of potassium bicarbonate. Optionally, approximately 0.2 grams of a flavonoid is added. This dry composition is dissolved in an amount of water as desired to produce a solution of desired tonicity. For example, if added to 100 ml of water it will provide a hypertonic solution, or, it added to 200 ml of water it will provide a hypotonic solution.

In another embodiment of the dry composition of the invention, approximately 5.8 grams of the dry formulation includes approximately 5 grams of magnesium ascorbate, and approximately 0.15 grams of potassium bicarbonate. Optionally, approximately 0.2 grams of quercetin or approximately 0.05 grams of bromelain is added to the composition. Optionally, other compounds such as flavorants may be added up to 5% by weight of the dry composition.

The dry compositions of the invention are combined with water, by dissolving a dry composition that is from approximately 50% to approximately 98% by weight of magnesium ascorbate, and from approximate 2% to approximately 50% by weight of potassium bicarbonate. The pH of the solutions is from approximately 6.0 to approximately 8.0, and in one embodiment has a pH of approximately 6.8 to approximately 7.1. The resulting solution contains from approximately 2.9% to approximately 5.68% by weight magnesium ascorbate and approximately 0.025% to approximately 0.7% by weight of potassium bicarbonate.

An embodiment of one liter of an isotonic solution of the invention contains approximately 50 grams of magnesium ascorbate and approximately 2 grams of potassium bicarbonate mixed with 1 liter of sterilized or purified water, at a pH of from approximately 6 to approximately 8. In one embodiment the pH of the solution is from approximately 6.5 to approximately 7.5. Optionally, 2 grams of quercetin is added to the solution rendering it hypertonic. For a hypotonic solution, this dry composition is dissolved in as much as 2 liters of water.

The slightly hypertonic solutions of the invention cleanse and reduce the inflammation in the subject's nasal area. While not wishing to be bound by any theory, the solutions of the invention will contain more dissolved solutes relative to the concentration of solutes in the cells of the nose and sinuses of the subject into which they are introduced, causing water to flow from the cells, thus reducing inflammation and opening closed nasal and sinus passages, as well as cleansing. An isotonic balance will be reached, and the magnesium of the composition will contact the cells of the subject, reducing any allergic response. The hypotonic solution adds water to the nasal cavity, thus aiding the removal of allergens and mucus from the nasal cavity, and diluting the concentration of allergens in the nasal cavity, causing mucus to be more fluid and more easily drained from the nasal cavity.

The compositions and solutions of the invention may include additional components physiologically compatible with internal use in humans and animals, including, but not limited to moisturizers, flavorants, oils such as eucalyptus oil, oregano oil, or rosemary oil and herbal extracts, to inhibit microbial and fungal growth, and provide additional comfort and palatability for the subject. In addition, an effective amount of preservatives such as benzalkonium chloride, or disodium phosphate, may be added to the dry compositions or solutions of the invention to enhance shelf life.

Nasal and sinus symptoms that may be treated by the compositions and methods of the invention include, but are not limited to, nasal allergy symptoms, nasal congestion, nasal inflammation, sinusitis, sinus infection, catarrh, rhinitis, allergic rhinitis, common cold, and postnasal drip. Methods of using the present compositions to cleanse nasal and sinus cavities of a subject and to reduce the symptoms of nasal and sinus irritation including inflammation and allergic reactions, are also provided. For example, hypertonic solutions may be used to reduce the symptoms of sinusitis in a subject in need of such treatment, or in cases of swollen and inflamed mucus membranes in allergy sufferers and allergic rhinitis, to reduce the inflammation of the nasal epithelium and the allergic reaction of mast cells. In addition to cleansing, i.e. removing irritants from the nasal and sinus cavities, the compositions of the invention may also be used to control, reduce, or inhibit the growth or the number of a variety of organisms that locate in the nose and sinuses, including fungi and bacteria. In addition to the anti-fungal activity of potassium bicarbonate, natural extracts such as oregano oil are known to have anti-bacterial effects.

The exact doses of the solutions containing the compositions of the invention are determined by the extent of the irritation of the nose and sinuses of the individual subject, and by the results obtained. For example, if a single application of solution results in sufficient relief, no additional administration needs to be performed. If a single application is insufficient, or the relief lessens, the subject may administer additional doses. It symptoms of irritation worsen, the subject discontinues administration of the solution, and may be advised to see a medical professional. The compositions of the invention are compatible with human and animal physiology. Alternatively, a medical professional can determine the appropriate dosage and timing of administration of the solution to a subject in his/her care.

The compositions of the invention can be provided in commerce in dry or liquid form. Dry compositions of the invention may be provided in single doses or in bulk, in sterile packaging for mixing with appropriate amounts of water, according to instructions provided with the packages. Compositions in liquid form, may be provided in sterile containers for administering to a subject, for example using a dropper. Alternatively, the compositions may be provided in a dispenser that provides a desired dosing, such as a pump nasal spray dispenser or aerosolized. Additionally, the solutions may be vaporized, for example rising a nebulizer, for inhalation by the subject.

It will be apparent to those of ordinary skill in the art that variations and alternative embodiments may be made given the foregoing description. Such variations and alternative embodiments are accordingly considered within the scope of the present invention.

1. A liquid nasal and sinus wash composition comprising magnesium ascorbate.

2. The wash composition of claim 1, further comprising a bicarbonate salt.

3. The composition of claim 2, wherein the bicarbonate salt is potassium bicarbonate.

4. The composition of claim 2, further comprising a flavonoid.

5. The composition of claim 4, wherein said flavonoid is quercetin or bromelain,

6. A dry composition for preparing a nasal and sinus wash comprising approximately 50% by weight to approximately 98% by weight of magnesium ascorbate, and from approximately 50% by weight down to approximately 2% by weight of a bicarbonate salt.

7. The dry composition of claim 6, wherein said bicarbonate salt is potassium bicarbonate.

8. The dry composition of claim 6, further comprising up to approximately 5% by weight of a flavonoid.

9. The dry composition, of claim 8, wherein said flavonoid is quercetin or bromelain.

10. A nasal and sinus wash solution comprising magnesium ascorbate and water.

11. The nasal and sinus wash solution of claim 10, further comprising potassium bicarbonate.

12. The nasal and sinus wash solution of claim 11, wherein the solution comprises approximately 2.9% to approximately 5.68% by weight of magnesium ascorbate, and from, approximately 0.025% to approximately 0.7% by weight of potassium bicarbonate.

13. The solution of claim 11, having a pH of from approximately 6.0 to approximately 8.0.

14. The solution of claim 11, having a pH of from approximately 6.8 to approximately 7.1.

15. The solution of claim 11, containing approximately 50 grants of magnesium ascorbate and approximately 2 grams of potassium bicarbonate in 1 lifer of sterilized or purified water.

16. The solution of claim 11, further containing approximately 2 grams of quercetin.

17. The solution of claim 11, wherein the solution is isotonic relative to human or animal cells.

18. The solution of claim 11, wherein the solution is hypertonic relative to human or animal cells.

19. The solution of claim 11, wherein the solution is hypotonic relative to human or animal cells.

20. A method of cleansing nasal and sinus cavities and reducing symptoms of nasal and sinus irritation, including inflammation, in a subject, comprising administering an effective amount of the solution of claim 11 into the nasal and sinus cavity of a subject.
